(19) United States
(12) Patent Application Publication
(10) Pub. No.: US 2024/0352467 A1
(43) Pub. Date: Oct. 24, 2024

(54) CHIMERIC COMPLEX AND THERAPEUTIC USES THEREOF

(71) Applicant: UNIVERSITA' DEGLI STUDI DI TORINO, Torino (IT)

(72) Inventors: Daniela TAVERNA, Chieri (Torino) (IT); Lorena QUIRICO, Venaria Reale (Torino) (IT); Francesca ORSO, Torino (IT)

(21) Appl. No.: 18/687,370

(22) PCT Filed: Aug. 30, 2022
(86) PCT No.: PCT/IB2022/058099

§ 371 Date: Feb. 28, 2024

(30) Foreign Application Priority Data
Aug. 31, 2021 (IT) 102021000022610

(51) Int. Cl. C12N 15/115; A61P 35/04

(52) U.S. Cl. CPC C12N 15/115; A61P 35/04; C12N 2310/113; C12N 2310/13; C12N 2310/16; C12N 2310/3519

(57) ABSTRACT

A chimeric complex including a nucleic acid aptamer directed against the tyrosine kinase receptor axl in combination with an oligonucleotide containing a DNA sponge directed against miR-214 is provided. Use of the chimeric complex for treatment of tumors is also provided. The treatment inhibits or reduces the onset and/or progression of tumor metastases.

The present invention falls within the field of therapeutic treatments of tumor diseases, particularly solid tumors, more particularly solid tumors expressing the oncogene axl.

Tumor diseases are typically characterized by progression through successive, increasingly severe stages. In an initial stage, normal cells, as a result of genetic modifications, begin to proliferate abnormally in a microenvironment consisting of stromal cells embedded in a remodelled extracellular matrix infiltrated by immune cells. Cancer cells that acquire the ability to invade adjacent tissues, intravasate, move through the vascular system, stop in the capillaries and extravasate into the surrounding tissue parenchyma give rise to distant metastases. Since metastatic spread is responsible for over 90% of cancer-related deaths, a great effort in the field of clinical and pharmacological research is aimed at identifying appropriate therapies which allow metastatic development to be stopped or at least slowed down.

In recent years, miRNAs, i.e., small non-coding RNAs acting as negative post-transcriptional regulators for their target genes, have been shown to be involved in tumor biology. In particular, in a vast majority of cases, the formation and progression of tumor disease was found to be associated with aberrant expression of certain miRNAs, and this finding is supported by growing emerging evidence. The first paper on the role of miRNAs in cancer appeared in 2002 and referred to the consequences of miR-15 and miR-16 deletion in chronic lymphocytic leukemia (CLL) (Calin, GA, et al., (2002) “Frequent deletions and down-regulation of micro-RNA genes miR15 and miR16 at 13q14 in chronic lymphocytic leukemia”; Proc Natl Acad Sci 99: 15524-15529). Over 32,500 studies have been published since then. Among the plurality of miRNAs identified so far, let-7 miRNAs, miR-29 family, miR-34 and miR-148b were shown to act as suppressors of “tumors or metastases”, whereas the miRNA 17-92 cluster, miR-21, miR-10b and miR-214 were shown to play a role in promoting tumor growth or spread, depending on the tumor context. In particular, recent studies have shown that miR-148b controls breast cancer progression by coordinating a large number of target molecules, including ITGα5 integrin, its downstream actors ROCK1 and PIK3CA/p110a (Cimino, D, et al. (2013) “miR148b is a major coordinator of breast cancer progression in a relapse-associated microRNA signature by targeting ITGA5, ROCK1, PIK3CA, NRAS, and CSF1”, FASEB J 27: 1223-1235) and the cell adhesion molecule ALCAM (Penna, E, et al. (2013) “miR-214 coordinates melanoma progression by upregulating ALCAM through TFAP2 and miR-148b downmodulation”, Cancer Res 73: 4098-4111). Furthermore, miR-148b expression was shown to be negatively regulated by the pro-metastatic miR-214, thus suggesting that miR-148b acts antagonistically in controlling the spread of breast cancer and melanoma (Orso, F, et al. (2016) “miR-214 and miR-148b Targeting Inhibits Dissemination of Melanoma and Breast Cancer”; Cancer Res 76: 5151-5162).

Due to the role of miRNAs in cancer progression, cancer therapies based on their use have been developed.

Dettori D, Orso F, Penna E, et al. (2018) “Therapeutic Silencing of miR-214 Inhibits Tumor Progression in Multiple Mouse Models”. Mol Ther.; 26 (8): 2008-2018 proposes miR-214 as a promising target for anti-metastatic therapies and describes an anti-miR-214 oligonucleotide capable of inhibiting the pro-metastatic functions of miR-214.

International patent application WO2012/049108 discloses a nucleotide aptamer capable of binding to and inhibiting the tyrosine kinase receptor axl. As is well known, this receptor is expressed at high levels by several types of tumors, including melanoma and breast tumor, while it is poorly expressed in normal cells.

International patent application WO2021/044282 discloses a chimeric complex formed by the anti-axl nucleotide aptamer of WO2012/049108 associated with an miRNA through complementary sticky sequences.

However, in the field of anticancer therapies there is a strong need to constantly develop new therapeutic tools that will increase the effectiveness and/or specificity and/or reduce the side effects of existing therapies.

There is also an ongoing need to provide new therapeutic strategies which are suited for targeting the progression of tumor diseases, thus making it possible to prevent, slow down and/or inhibit the onset of tumor metastases, while hampering the onset of any adverse side effects.

The need to provide alternative therapeutic strategies, which is felt to be of fundamental importance in the field of anticancer therapies, is also caused by the fact that some patients may develop resistance to certain anticancer therapies, such as chemotherapy. This problem is particularly felt in the case of tumors expressing the tyrosine kinase receptor axl, whose presence on the surface of tumor cells often leads to the onset of resistance.

In order to meet these and other needs, the present invention provides the chimeric complex as defined in the appended claim 1, and the pharmaceutical composition comprising it.

Additional features and advantages of the invention are defined in the dependent claims, which form an integral part of the description.

As will be apparent from the following detailed description, the present invention provides a chimeric complex which is defined by a combination of features capable of providing an effective antitumor activity, in particular anti-metastatic activity, accompanied by significant selectivity of action.

In the context of the present description, the term “chimeric complex” refers to a macromolecular complex comprising: i) an aptamer directed against the tyrosine kinase receptor axl (for the sake of brevity hereinafter referred to as “anti-axl aptamer”), wherein the aptamer is an RNA aptamer or a DNA aptamer, and ii) a DNA sponge sequence directed against miR-214 (for the sake of brevity hereinafter referred to as “miR-214sponge”), wherein the above two components are capable of exerting different actions.

The chimeric complex of the invention is sometimes hereinafter referred to as “axl-miR-214sponge”.

Generally, the term “aptamer” indicates a single stranded oligonucleotide molecule capable of binding to a certain target molecule, for example a cell transmembrane protein, with high affinity and selectivity. The axl receptor, i.e., the target of the anti-axl aptamer of the chimeric complex according to the invention, is known to be aberrantly expressed on the surface of a large prevalence of different tumor cells, where it exerts oncogenic activity.

The anti-axl aptamer of the chimeric complex according to the invention comprises, from the 5′ end to the 3′ end:
(i) a single stranded nucleic acid sequence (RNA or DNA) capable of binding to the tyrosine kinase receptor axl;
(ii) a linker element consisting of a linear unsubstituted alkyl chain containing 4 to 20 carbon atoms; and
(iii) a first sticky sequence consisting of a single stranded nucleic acid sequence (RNA or DNA) 15 to 20 bases in length.

The linker element is a linear unsubstituted alkyl chain containing 4, 5, 6, 7, 8, 9, 10, 11, 12, 13, 14, 15, 16, 17, 18, 19, or 20 carbon atoms. The linear unsubstituted alkyl chain preferably contains 6 to 18 carbon atoms, more preferably 12 carbon atoms.

In one embodiment of the invention, the single stranded nucleic acid sequence capable of binding to the axl aptamer is the “GL21.T” RNA sequence described in WO2012/049108 (SEQ ID NO:1).

In another embodiment of the invention, the first single-stranded sticky nucleic acid sequence of the anti-ax aptamer is the RNA sequence of SEQ ID NO:2.

With reference to both of the above-mentioned embodiments, the RNA sequence of SEQ ID NO: 1 and/or the RNA sequence of SEQ ID NO:2 can be replaced by the respective modified DNA sequence in which each U (uridine) of SEQ ID NO:1 and/or SEQ ID NO:2 is replaced by a dU (deoxyuridine).

In fact, from WO20124095A1 and from the paper by Amero P. et al. (2021), J. Am. Chem. Soc. 143, 20, 7655-7670 the conversion of an RNA aptamer into a modified DNA aptamer is known to provide prolonged stability and improved antitumor activity.

The chimeric complex according to the invention also includes a second element consisting of an oligonucleotide containing a DNA sponge directed against miR-214 (for the sake of brevity referred to as “miR-214sponge”). This comprises, from the 5′ end to the 3′ end:
(iv) a binding sequence consisting of a single stranded DNA containing a plurality of miR-214 binding regions interspersed with one or more linking regions, and
(v) a second sticky single stranded DNA sequence having the same base length as the first sticky single stranded nucleic acid sequence identified above in (iii) and complementary thereto.

In one embodiment of invention, the binding sequence identified above in (iv) is SEQ ID NO: 3. However, in other embodiments of invention, the binding sequence SEQ ID NO:3 is modified in the nucleotide sequence of the miR-214 binding regions, the number of miR-214 binding regions (e.g., 2, 3 or 4), and/or the length of the linking regions.

Non-limiting examples of modified binding sequences are SEQ ID NOs: 7-28 shown below.

In one embodiment of invention, the second sticky sequence of the miR-214sponge identified above in (v) is the sequence SEQ ID NO:4.

As mentioned above, the first and second sticky sequences of the anti-axl aptamer and the miR-214sponge, respectively, have the same length in terms of bases and are complementary to each other. As a result, the annealing of these two nucleotide sequences allows the association of the aptamer with the oligonucleotide, thereby the formation of the chimeric complex.

The present invention is based on the results obtained by the inventors in the experimentation and research activities described in the following experimental section.

In short, in vitro studies carried out by the present inventors with axl-expressing breast cancer and melanoma cells revealed that the use of the chimeric complex according to the invention results in a significant reduction in migration, invasiveness, and transendothelial migration of tumor cells, while increasing the direct targets of the TFAP2γ and ITGα3 miR-214 and decreasing the indirect targets of the ALCAM and ITGα5 miR-214, thus proving to be able to therapeutically affect the axis formed by miR-214 and miR-148b. In fact, miR-148b is a small anti-metastatic RNA that is inhibited by miR-214, which is capable of silencing ALCAM and ITGα5. Furthermore, treatment with the chimeric complex of the invention inhibits the formation and growth of breast cancer cell mammospheres in vitro. The effects mentioned above only occur in cells expressing axl on their cell surface, not in cells that do not express this receptor. In addition, the inventors found that while the anti-axl aptamer alone can also block the metastatic characteristics of tumor cells expressing axl. the chimeric complex of the invention has a significantly higher effect than the anti-axl aptamer alone, suggesting a combined activity of the anti-axl aptamer and the miR-214sponge.

Lastly, when injected into a primary tumor mass or systemically injected into the tail vein in mice, the chimeric complex according to the invention showed a strong ability to block the metastatic process in vivo.

In the light of the above, the chimeric complex according to the invention represents an innovative therapeutic tool in the oncological field, which is particularly effective in counteracting (i.e., inhibiting or reducing) tumor invasiveness and metastatic progression, and at the same time characterized by a considerable reduction of adverse side effects thanks to the particular selectivity of action of the aptamer which is capable of mediating the specific binding of said complex to the axl-positive target tumor cell and its internalization. In addition, the presence of the miR-214sponge further increases the selectivity of action, making the chimeric complex of the invention particularly effective against solid tumors in which both axl and miR-214 are overexpressed, such as melanoma, pancreatic tumor, stomach tumor, prostate tumor, lung tumor, breast tumor (especially triple-negative breast tumor), ovary tumor.

Preferably, the chimeric complex according to the invention is nuclease-resistant.

For this purpose, one or more pyrimidine bases of the chimeric complex according to the invention is/are preferably replaced with the corresponding 2′-fluoropyrimidine (replacement of all pyrimidine bases with the corresponding 2′-fluoropyrimidine is particularly preferred). It is also preferable for one or more purine bases of the chimeric complex to be replaced with the corresponding 2′-O-methylpurine. Replacement of purines with the corresponding 2′-O-methylpurine is particularly preferred in the two sticky sequences.

In one embodiment of the invention, in addition to the nucleotide replacements described above, or alternatively thereto, in order to increase the nuclease-resistance, the 3′ end of the first sticky sequence (e.g., SEQ ID NO: 2) and/or the 3′ end of the second sticky sequence (e.g., SEQ ID NO: 4) in the chimeric complex of the invention is/are locked by conjugation with a biotin molecule.

Further nucleotide sequence modifications suitable for conferring nuclease resistance include, for example, but are not limited to, the addition of 2′-amino (2′-NH₂) ribose, monothiophosphates or thiophosphates, modifications to the phosphodiester bond (phosphorothioates and methylphosphonates), the use of phosphoramidates, 2′-O-alkyl ribonucleotides, replacement with locked nucleic acids (LNA) or peptide nucleic acids (PNA).

In another embodiment of the invention, the chimeric complex of the invention additionally comprises polyethylene glycol (PEG) or cholesterol in order to decrease renal clearance.

The embodiments of the invention described above can be combined with one another and the various achievable combinations fall within the scope of the invention.

Thanks to its targeted anti-tumor, in particular anti-metastatic activity, the chimeric complex according to the invention is suitable for use in the therapeutic treatment of tumor diseases, preferably tumor diseases characterized by deregulated activity of the tyrosine kinase receptor axl, more preferably tumors in which both axl and miR-214 are overexpressed.

Tumor diseases include, but are not limited to, melanoma, pancreatic tumor, stomach tumor, prostate tumor, lung tumor, breast tumor (especially triple-negative breast tumor), ovary tumor.

A pharmaceutical composition comprising the chimeric complex of the invention as defined above, in combination with at least one pharmaceutically acceptable carrier, excipient and/or diluent, is also within the scope of the invention.

According to the invention, the pharmaceutical composition is suitable for use in the above therapeutic medical applications relating to the chimeric complex.

The pharmaceutical composition of the present invention can be formulated into any suitable dosage form, for example, for subcutaneous, intravenous, intra-arterial, intraperitoneal, intramuscular, intranasal, or inhalatory administration.

In an alternative embodiment, the pharmaceutical composition according to the invention can be formulated into a dosage form suitable for local intra-tumor administration, for example by injection under computed tomography guidance.

Of course, the selection of suitable carriers, excipients and/or diluents is carried out depending on the desired form of administration and this selection is within the skills of those of ordinary skill in the art. The selection of the active principle dose and dosage regimen also falls within the skills of those of ordinary skill in the art, and the selection thereof depends on several factors, such as for example the age and weight of the patient, the type of tumor, the degree of progression of the disease, as well as the size of the tumor mass to be treated.

The invention is further described in the examples below, with reference to the accompanying drawings, wherein:

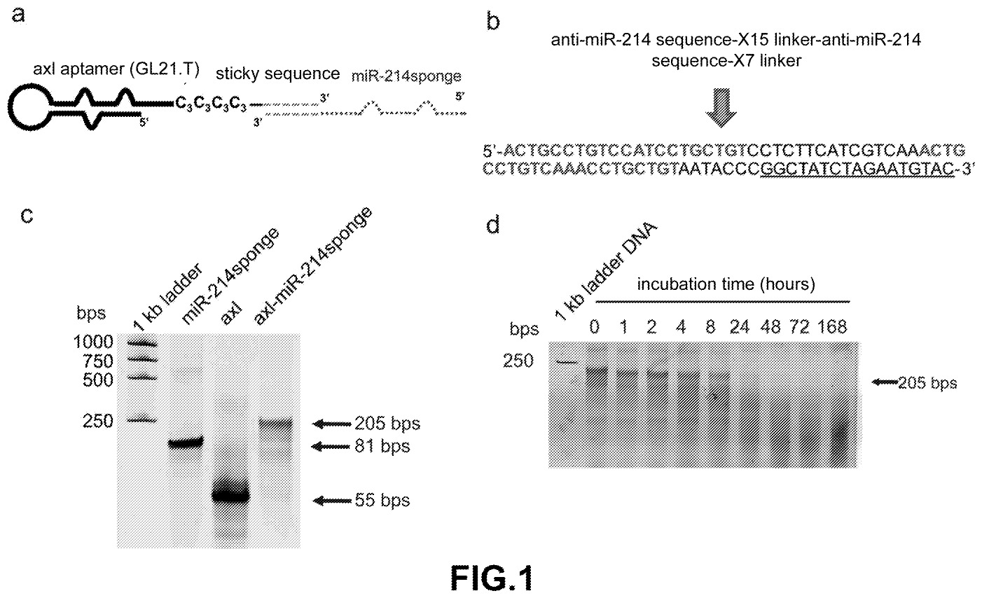

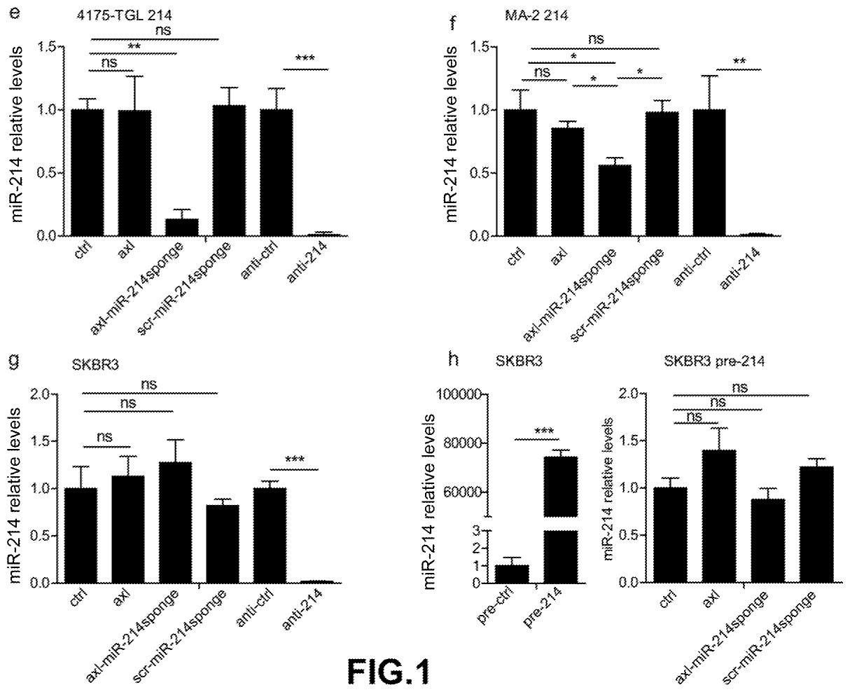

FIG. 1 shows the structure of the chimeric complex of the invention and its impact on miR-214 expression in axl-positive or axl-negative tumor cells. (a) A schematic representation of the chimeric complex according to the invention (“axl-miR-214sponge”). (b) miR-214sponge sequence. (c) An image depicting a polyacrylamide gel electrophoresis to check the correct formation of the chimeric complex. The first band corresponds to miR-214sponge, the second band to the anti-axl aptamer, and the third band to the chimeric axl-miR-214sponge complex according to the invention. (d) Images depicting non-denaturing polyacrylamide gel electrophoresis experiments showing the stability of the axl-miR-214sponge chimeric complex (4 μM) in 80% human serum at the indicated time points (h=hours). For (c-d), reference=1kb. (e-h) miR-214 relative expression levels obtained by qRT-PCR in 4175-TGL and MA-2 cells expressing axl and overexpressing miR-214, or in axl-negative SKBR3 cells, after treatment with the control solution (ctrl), the anti-axl aptamer alone, the axl-miR-214sponge of the invention, or the scr-miR-214sponge. Transfection with anti-miR-214 (anti-214) or pre-miR-214 (pre-214) or their respective controls (anti-ctrl or pre-ctrl) was also performed and the respective expression levels of miR-214 were analyzed. Results are shown as fold changes (mean±SD) versus controls (ctrl or anti-ctrl), normalized to the small nucleolar RNA U44 levels. Three independent experiments were performed in triplicate and a representative one is shown. Ns=not significant, *p<0.05, **p<0.01, ***p<0.001. SD=Standard Deviation.

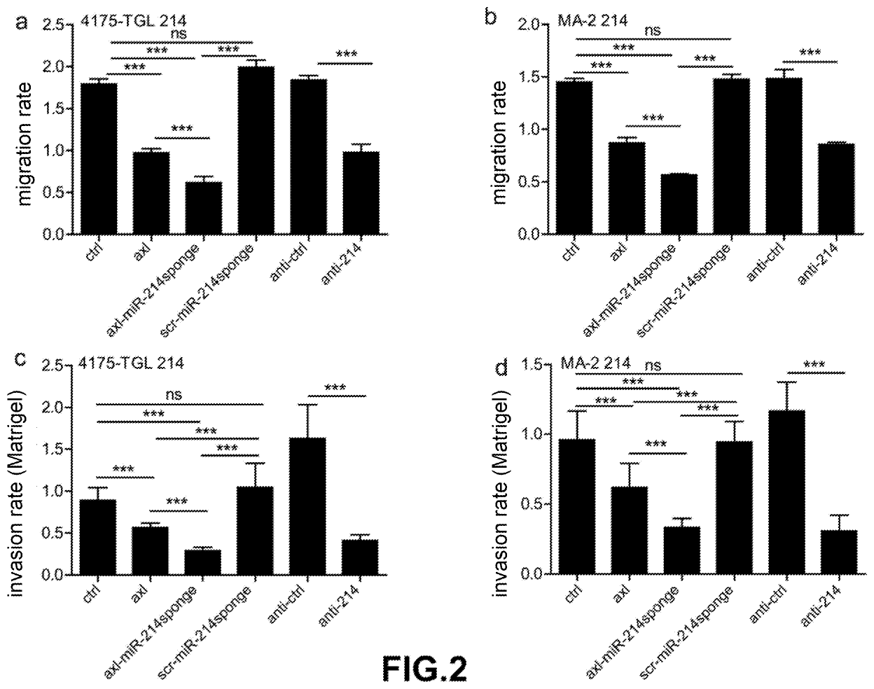

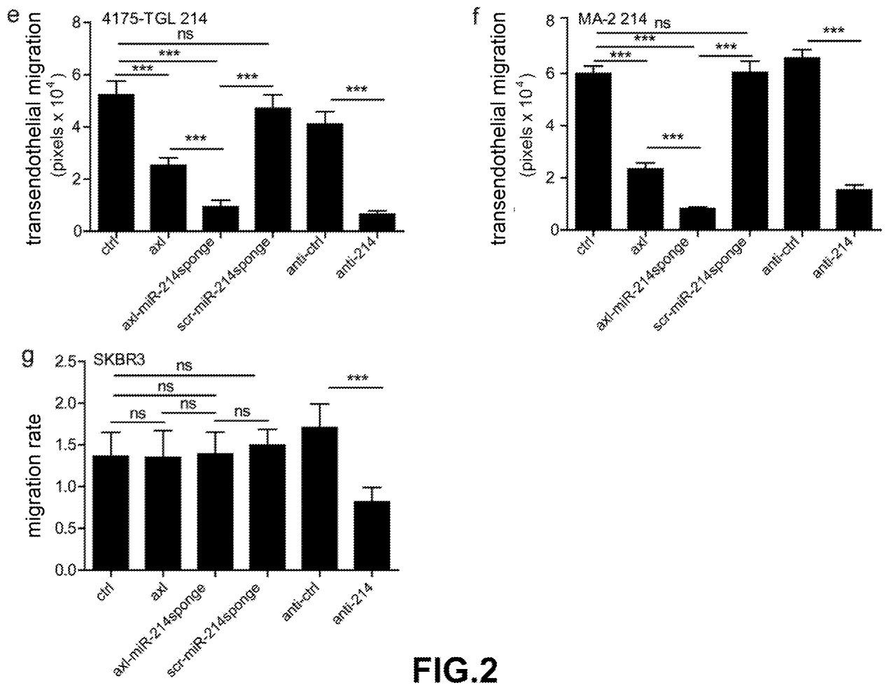

FIG. 2 shows that the chimeric complex of the invention is capable of inhibiting breast tumor and melanoma cell motility. (a-g) Transwell migration (a-b, g), invasiveness through Matrigel (c-d) or transendothelial migration through a HUVEC monolayer (e-f) assays for 4175-TGL and MA-2 cells expressing axl and overexpressing miR-214, and for axl-negative SKBR3 cells, treated with the ctrl solution, the anti-axl aptamer alone, or the axl-miR-214sponge or scr-miR-214sponge complex. Transfection with anti-miR-214 (anti-214) and its control (anti-ctrl) was also performed. The results in the graphs are expressed as the ratio of the mean±SEM of the area covered by the migrated/invasive cancer cells vs plated cells (a-d, g) or as the mean±SEM of the area (pixels) covered by the transmigrated cells (e-f). At least two independent experiments (in triplicate) were carried out, and representative results thereof are shown. ns=not significant; *p<0.05, **p<0.01, ***p<0.001; SEM=Standard Error of the Mean.

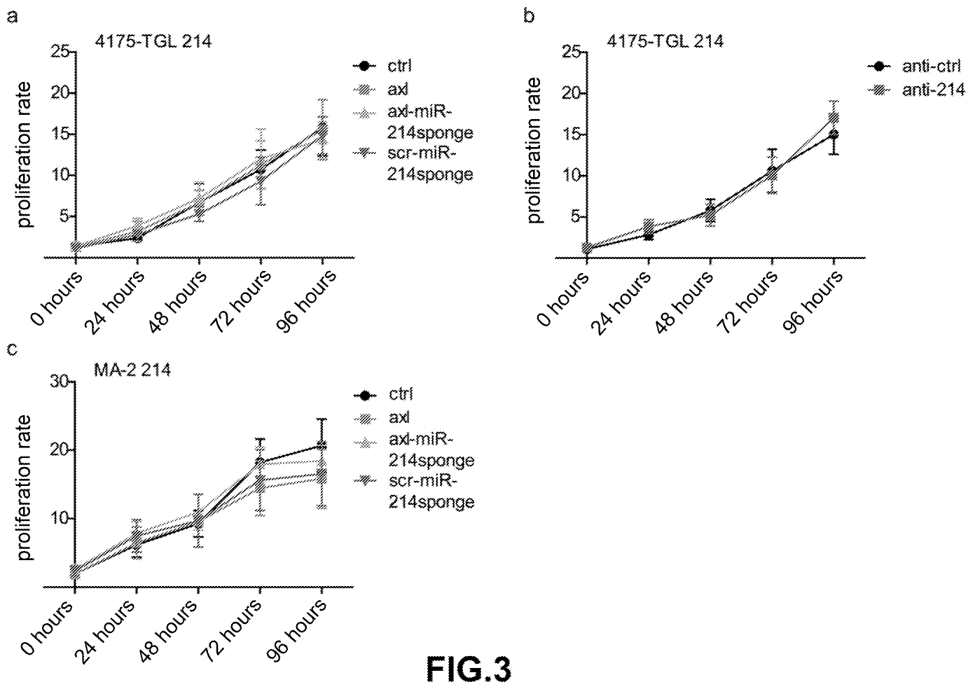

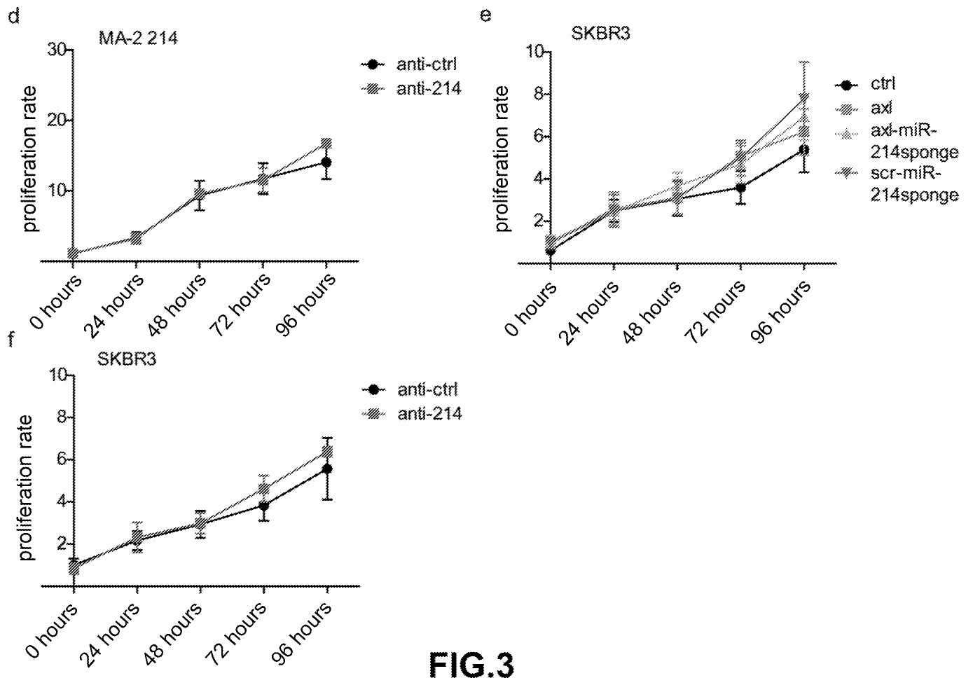

FIG. 3 shows that the chimeric complex of the invention has no effects on tumor cell growth. (a-f) Growth assays for 4175-TGL and MA-2 cells expressing axl and overexpressing miR-214, and for axl-negative SKBR3 cells, treated with the ctrl solution, the anti-axl aptamer alone, or the axl-miR-214sponge or scr-miR-214sponge complex. Results are shown as the mean±SD of the ratio between growth and number of plated cells, measured as the optical density at 0-96 hours (h). At least 2 independent experiments were performed in triplicate and representative results thereof are shown. SD=Standard Deviation.

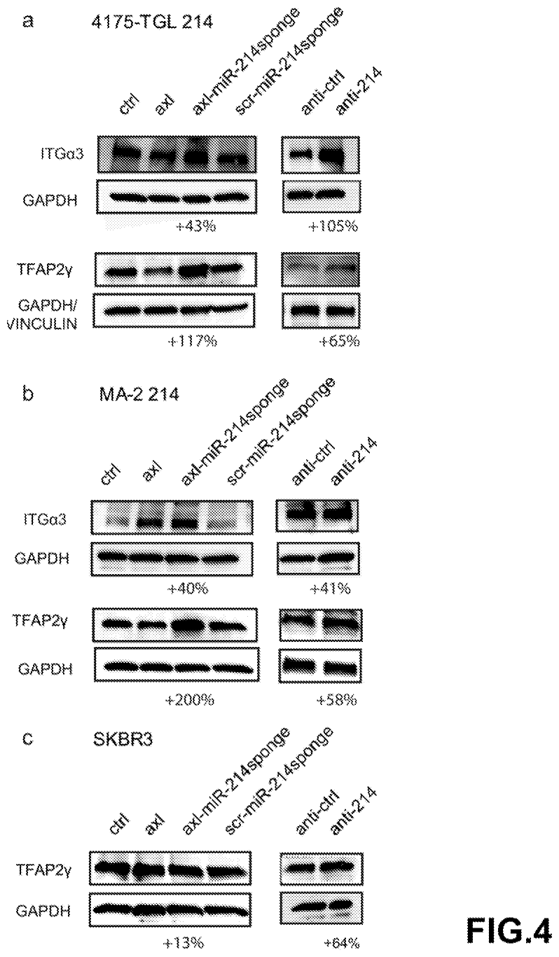

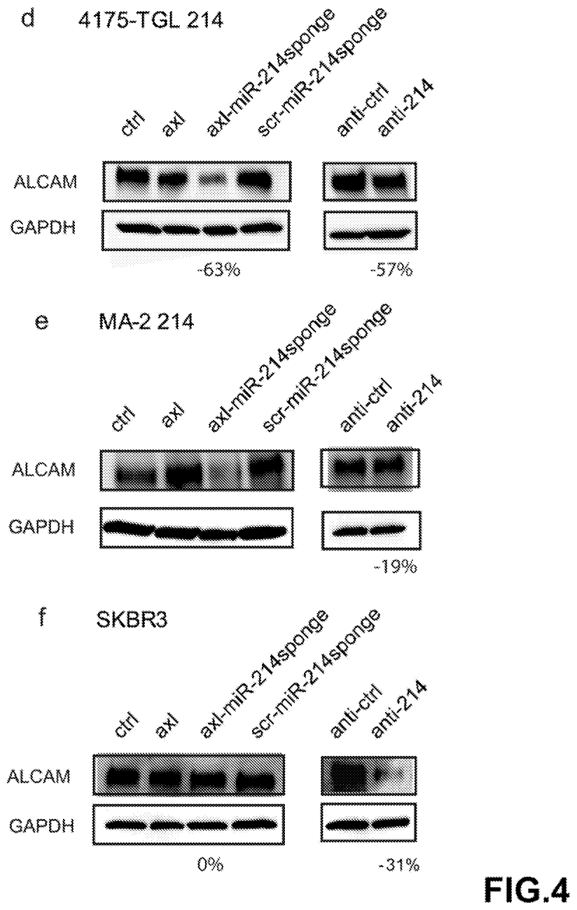

FIG. 4 shows that the chimeric complex of the invention modulates the expression of miR-214 targets in axl-expressing breast tumor and melanoma cells. (a-f) Western blot analysis of the direct miR-214 targets ITGα3 and TFAP2γ or of the indirect target ALCAM in 4175-TGL and MA-2 cells expressing axl and overexpressing miR-214 (a-b, d-e), or in axl-negative SKBR3 cells, treated with the control solution, the anti-axl aptamer alone, or the axl-miR-214sponge or scr-miR-214sponge complex. Transfection with anti-miR-214 (anti-214) and its control (anti-ctrl) was also performed. Proteins were collected after 48 hours. Protein variations were calculated with reference to the anti-axl or anti-ctrl aptamer, normalized to loading controls (Vinculin or GAPDH), and expressed as percentages (%). At least three independent experiments were performed, and representative results thereof are shown. For the top and left panels, a and d, GAPDH is the same because the blots were incubated with anti-ITGα3 antibody, striped, and incubated with anti-ALCAM antibody.

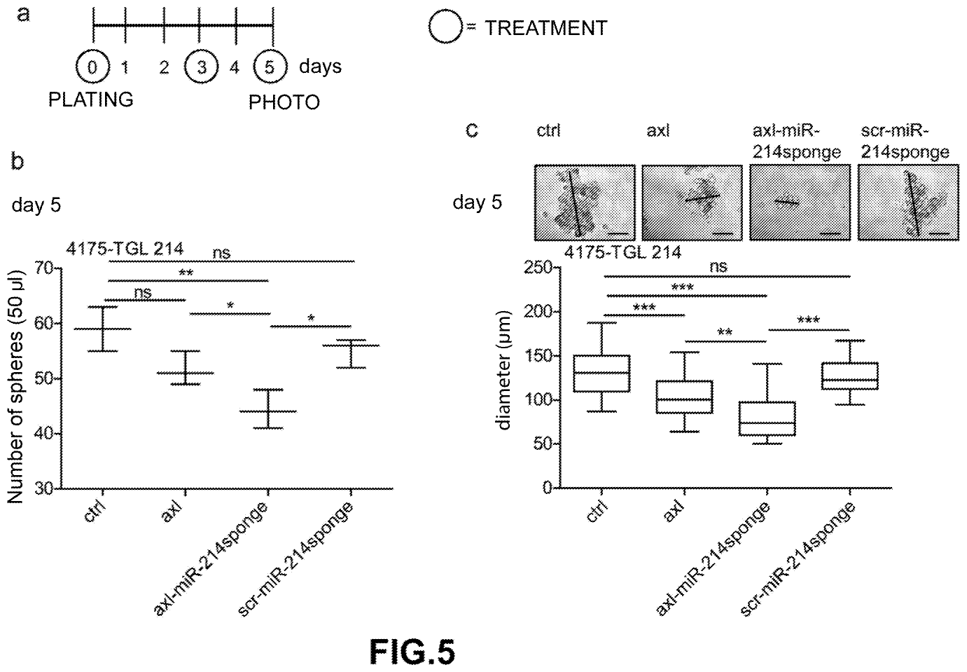

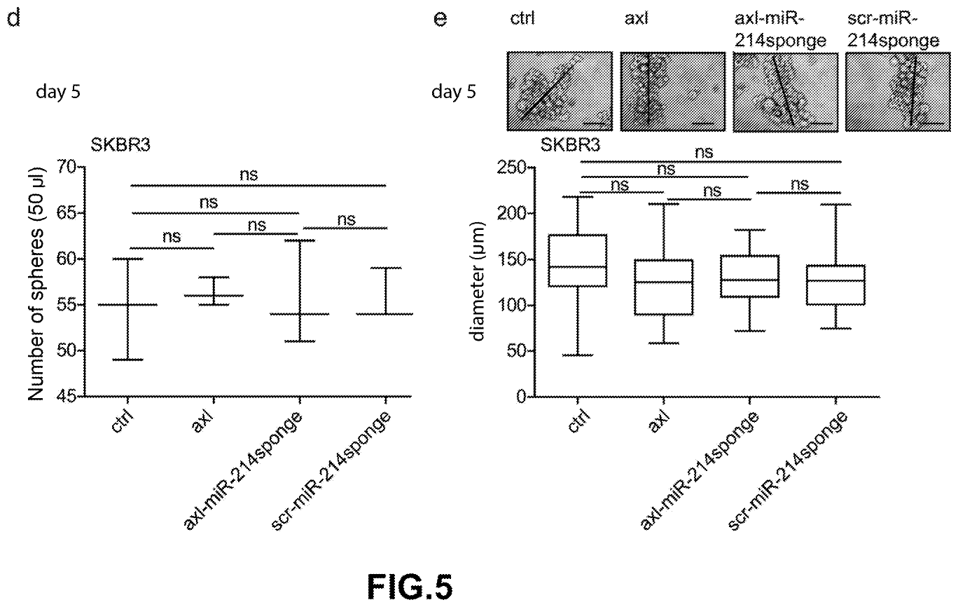

FIG. 5 shows that the chimeric complex of the invention reduces the quantity and size of mammospheres derived from axl-positive breast tumor cells, but not from axl-negative breast tumor cells. (a) Schematic representation of the protocol: 4175-TGL breast tumor cells expressing axl and overexpressing miR-214 or SKBR3 axl-negative breast tumor cells were plated (Day 0), maintained in suspension and treated with the ctrl solution, the anti-axl aptamer alone, or the axl-miR-214sponge or scr-miR-214sponge complex. Treatments were repeated on days 3 and 5. (b-d) On day 5, the size and number of spheres were assessed under a microscope. Both the number and size of the spheres are shown as the mean±SEM of the length of the spheres (μm). (b,d) Plots depicting the number of 4175-TGL 214 or SKBR3-derived mammospheres in a volume of 50 μl of medium on day 5, shown as the mean±SEM. (c-e) Top: Photographs representing mammospheres. Bottom: plots relating to the size of the spheres, shown as the mean±SEM of the length of the spheres (μm); the black lines correspond to size measurements. At least two independent experiments (in triplicate) were carried out, and representative results thereof are shown. ns=not significant; *p<0.05, **p<0.01, ***p<0.001; SEM=Standard Error of the Mean; Scale bar=25 μm (c-e).

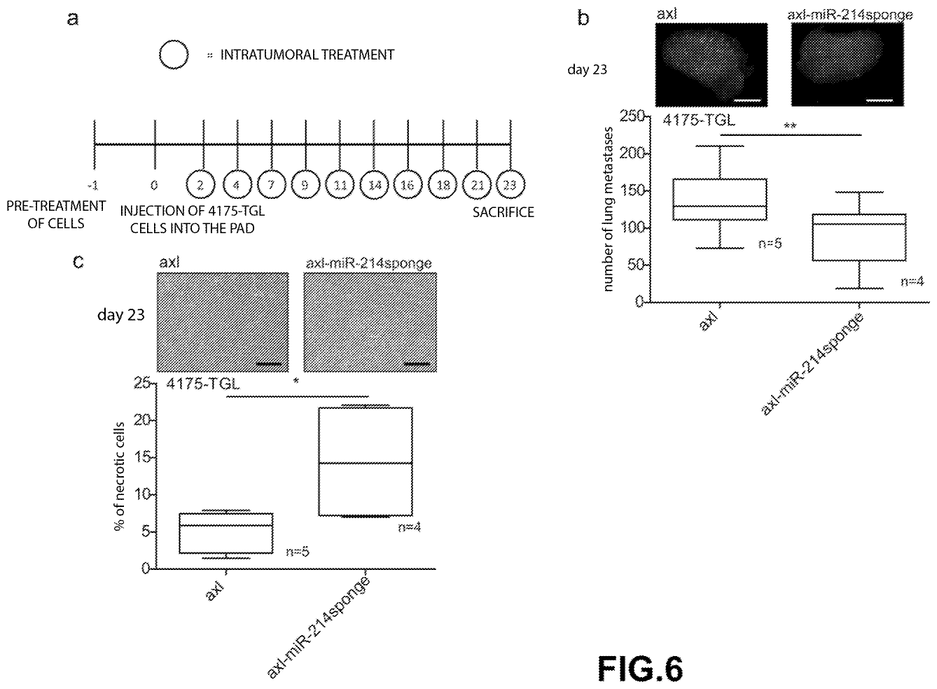

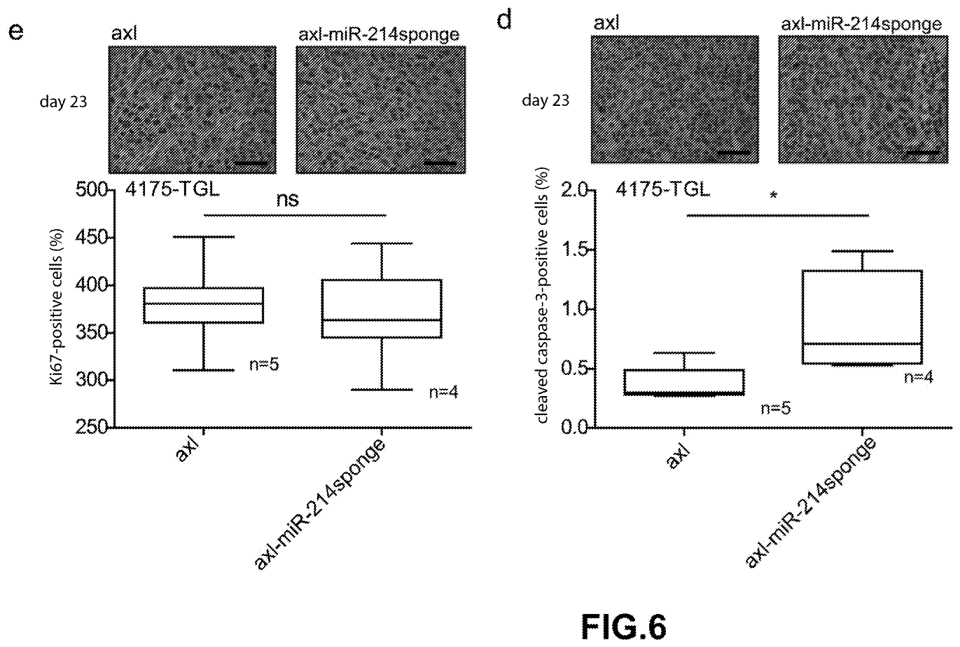

FIG. 6 shows that axl-miR-214sponge reduces metastasis and promotes necrosis and apoptosis in breast tumor xenografts. (a) Diagram of the experiment: 4175-TGL breast tumor cells were pretreated for 24 hours with either the anti-axl aptamer or the axl-miR-214sponge and then injected orthotopically into the mammary gland of NOD/SCID/IL2R_null mice (NSG). axl or axl-miR-214sponge was administered into the tumors starting on day 2 post-injection (3 treatments/week, 400 pmoles in 100 μl, 10 injections in total, as indicated), and the primary tumors were analyzed. (b) Images depicting the lungs are shown. The plot indicates the number of lung metastases, and the results are reported as the mean±SEM for the indicated number (n) of mice. (c) Primary tumor sections were stained with H&E and the necrotic areas were assessed: representative images are shown above the plots indicating the percentage (%) of the necrotic area vs the total area, shown as the mean±SEM for the indicated number (n) of mice. (d-e) Primary tumors were IHC stained with either a cleaved anti-Caspase-3 antibody (d) or KI67 (e), and the nuclei were counter-stained with hematoxylin (blue): representative images are shown above the plots indicating the percentage (%) of positive cells vs the total number of cells, shown as the mean±SEM for the indicated number (n) of mice (10 fields per mouse were assessed). IHC=immunohistochemistry. H&E=Hematoxylin & Eosin. Scale bar=100 μm (c) or 25 μm (d, e). ns=not significant; *p<0.05; **p<0.01; ***p<0.001. SEM=Standard Error of the Mean.

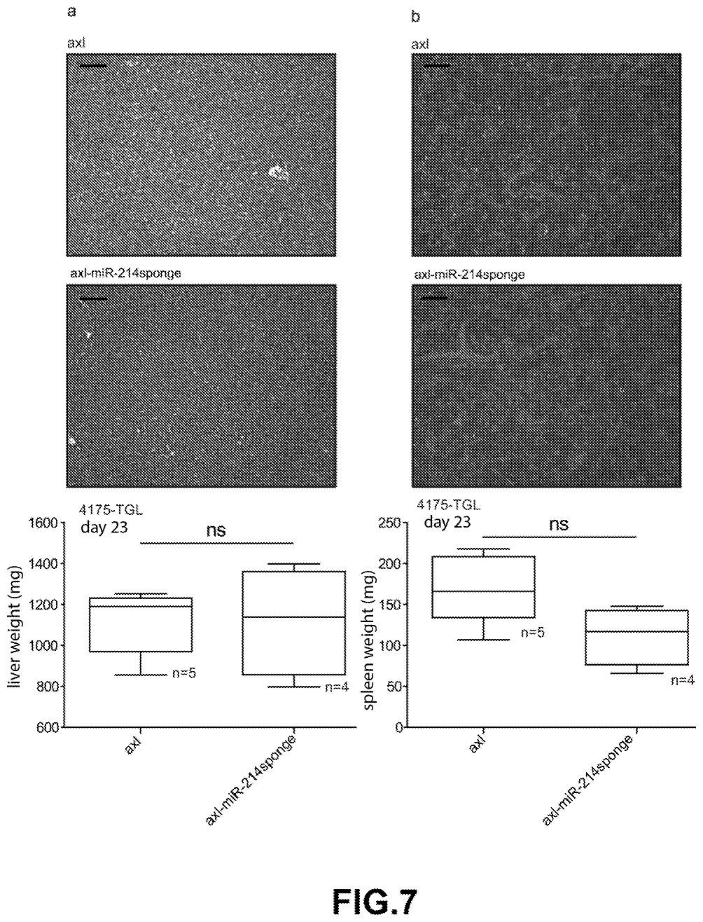

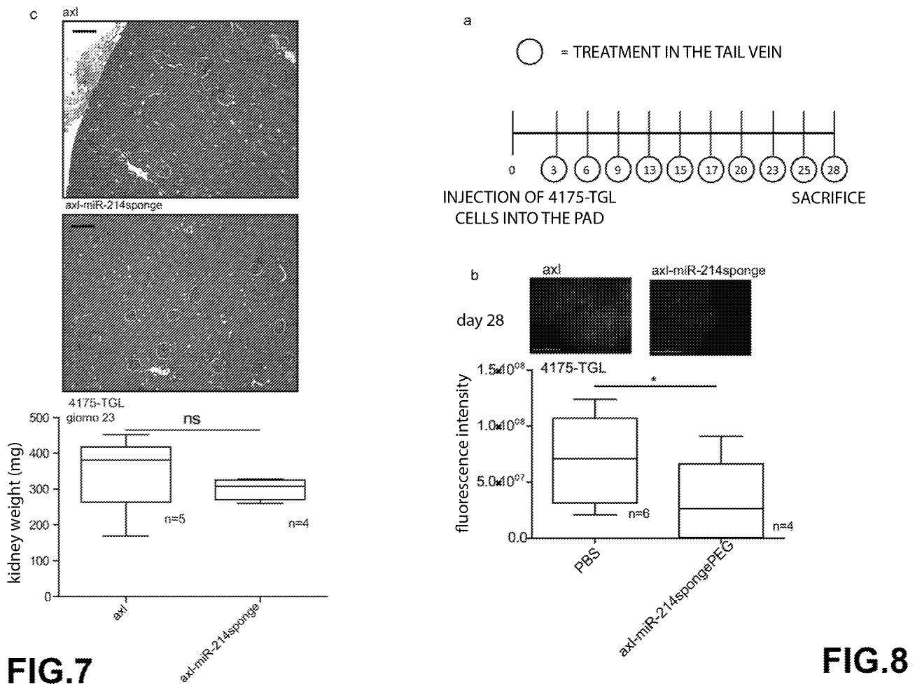

FIG. 7 shows that axl-miR-214sponge is not toxic to mice. (a-c) Mice were injected with 4175-TGL cells and treated as described in FIG. 6. The liver (a), spleen (b) and kidneys (c) were weighed on day 23 (the day the experiment ended) and the sections were stained with H&E. Photographs depicting the organs (scale bar=100 or 200 μm) are shown above the plots representing the mean±SEM of the weight of the organs for the indicated number (n) of mice. ns=not significant. SEM=Standard Error of the Mean; H&E=Hematoxylin & Eosin; mg=milligrams.

FIG. 8 shows that axl-miR-214sponge PEG prevents breast tumor spread in mice injected systemically. (a) Diagram of the experiment. 4175-TGL breast tumor cells were injected orthotopically into the mammary gland of NOD/SCID/IL2R_null mice (NSG), and PBS or the aptamer axl-miR-214sponge PEG was administered into the mouse tail vein starting on day 3 post-injection (3 treatments/week, 1600 pmoles in 100 μl, 10 injections in total, as indicated). (b) Images depicting the lungs and their metastases are shown. The plot indicates the fluorescence of lung metastases, and the results are reported as the mean±SEM for the indicated number (n) of mice. Scale bar=800 μm. ns=not significant; *p<0.05; **p<0.01; ***p<0.001. SEM=Standard Error of the Mean.

### EXAMPLES

### Materials and Methods

### Cell Cultures

MA-2 cells were kindly provided by L. Xu and R. O. Hynes (Xu, L, et al. (2008). Gene expression changes in an animal melanoma model correlate with aggressiveness of human melanoma metastases. Mol Cancer Res 6: 760-769.) and maintained as described in (Penna, E, Orso, F et al. (2011). “microRNA-214 contributes to melanoma tumour progression through suppression of TFAP2C”. EMBO J 30: 1990-2007; Penna, E, et al. (2013) “miR-214 coordinates melanoma progression by upregulating ALCAM through TFAP2 and miR-148b downmodulation”, Cancer Res 73: 098-4111). SKBR3 cells were purchased from the American Type Culture Collection (ATCC), while 4175-TGL cells were kindly provided by J. Massagué (Minn, AJ et al. (2005). Genes that mediate breast cancer metastasis to lung. Nature 436: 518-524) and maintained under standard culture conditions. HUVEC cells (human endothelial cells obtained from the umbilical cord) were kindly provided by M. F. Brizzi and maintained as described in (Penna, E, Orso, F et al. (2011). “microRNA-214 contributes to melanoma tumour progression through suppression of TFAP2C”. EMBO J 30: 1990-2007; Penna, E, et al. (2013) “miR-214 coordinates melanoma progression by upregulating ALCAM through TFAP2 and miR-148b downmodulation”, Cancer Res 73: 4098-4111).

### Reagents and Antibodies

The following were used as anti-miR inhibitors: Anti-miR miRNA Inhibitor Negative Control #1, Anti-miR miRNA Inhibitor hsa-miR-214 (AM12124) (Applied Biosystems). The following reagents were used for the analysis of miRNA expression levels: MicroRNA TaqMan®: Hsa-miR-214 ID 002306, U6 snRNA ID001973, U44 snRNA ID001904 (Applied Biosystems). Primary antibodies: anti-Cleaved Caspase-3 (Asp175) #9661 (Cell Signaling Technology), anti-KI67 ab15580 (Abcam), anti-ITGα5pAb RM10 kindly provided by G. Tarone (Molecular Biotechnology Center, University of Turin), anti-ITGα3 kindly provided by E. Turco (Molecular Biotechnology Center, University of Turin), anti-CD166/ALCAM mAb MOG/07 (Novocastra Laboratories), anti-TFAP2γ (6E4/4), anti-GAPDH pAb V-18, anti-ACTIN I-19 pAb (from Santa Cruz Biotechnology), anti-α-tubulin mAb B5-1-2 (Sigma). Secondary antibodies: HRP-conjugated goat anti-mouse IgG, goat anti-rabbit IgG (Santa Cruz Biotechnology).

### Transient Transfections and Production of Stable Cell Lines

In order to obtain miRNA transient expression and stable cell lines for the expression of the miRNAs, the cells were treated as described in ((Penna, E, Orso, F et al. (2011). “microRNA-214 contributes to melanoma tumour progression through suppression of TFAP2C”. EMBO J 30: 1990-2007; Orso, F, et al. (2016) “miR-214 and miR-148b Targeting Inhibits Dissemination of Melanoma and Breast Cancer”; Cancer Res 76: 5151-5162)5).

### Preparation of the Chimeric Complex

In order to generate an axl-miR-214sponge chimeric complex falling within the scope of the invention, an miR-214 sponge was conjugated with an anti-axl aptamer (“GL21.T”) (Cerchia et al. (2012). Targeting Axl with a high-affinity inhibitory aptamer. Mol Ther 20: 2291-2303), by annealing the sequences through their sticky ends.

The following sequences were used:

5′AUGAUCAAUCGCCUCAAUUCGACAGGAGGCUCAC 3′ (GL21.T binding
sequence capable of binding to the tyrosine kinase receptor axl, SEQ ID NO: 1);
(first sticky sequence, SEQ ID NO: 2)
5′GUACAUUCUAGAUAGCC 3′;
5′ACTGCCTGTCCATCCTGCTGTCCTCTTCATCGTCAAACTGCCTGTCAAACCTG
CTGTAATACCC 3′ (binding sequence with multiple anti-miR-214 sites, SEQ ID NO: 3);
(second sticky sequence, SEQ ID NO: 4)
5′GGCTATCTAGAATGTAC 3′;
(GL21.T scramble, SEQ ID NO: 5)
5′GGCGCUAGAACCUUCUAAGCGAAUACAUUACCGC 3′;
GL21.T-sticky (anti-axl aptamer):
(SEQ ID NO: 1-XXXX-SEQ ID NO: 2)
5′AUGAUCAAUCGCCUCAAUUCGACAGGAGGCUCACXXXXGUACAUUCUAGA
UAGCC 3′;
GL21.T scramble (scr aptamer):
(SEQ ID NO: 5-XXXX-SEQ ID NO: 2)
5′GGCGCUAGAACCUUCUAAGCGAAUACAUUACCGCXXXXGUACAUUCUAGA
UAGCC 3′;
miR-214sponge:
(SEQ ID NO: 6)
5′ACTGCCTGTCCATCCTGCTGTCCTCTTCATCGTCAAACTGCCTGTCAAACCTG
CTGTAATACCCGGCTATCTAGAATGTAC 3′.

In the anti-axl and scramble aptamer sequences “XXXX” represents the linear unsubstituted alkyl sequence having 4 to 2 carbon atoms acting as a linker.

### Alternative Sequences to SEQ ID NO:3

The following binding sequences may be used as alternatives to the binding sequence SEQ ID NO: 3:

(SEQ ID NO: 7)
5′ACTGCCTGTCTAACCTGCTGTAACTACCAATACCAAACTGCCTGTCA
ACCCTGCTGTCTTTTGT 3′
(SEQ ID NO: 8)
5′ACTGCCTGTCCGTCCTGCTGTCCCATCTATTTCCTTACTGCCTGTCC
ACCCTGCTGTTTAATCC 3′
(SEQ ID NO: 9)
5′ACTGCCTGTCCCTCCTGCTGTAACAACCTTCACCGAACTGCCTGTCC
ATCCTGCTGTATCATCC 3′
(SEQ ID NO: 10)
5′ACTGCCTGTCAATCCTGCTGTCACTAATTTCATCCAACTGCCTGTCA
CCCCTGCTGTCTATCCC 3′
(SEQ ID NO: 11)
5′ACTGCCTGTCCGTCCTGCTGTATCTTACTTCATCAAACTGCCTGTCC
CTCCTGCTGTCCCCCCC 3′
(SEQ ID NO: 12)
5′ACTGCCTGTCCGTCCTGCTGTCGTATCCTACCTTAAACTGCCTGTCC
CACCTGCTGTATCCCCC 3′
(SEQ ID NO: 13)
5′ACTGCCTGTCCATCCTGCTGTCATTACTCCCCTTATACTGCCTGTCA
ATCCTGCTGTTATTCCC 3′
(SEQ ID NO: 14)
5′ACTGCCTGTCCATCCTGCTGTCTCCCGTTTCTTTTTACTGCCTGTCC
CTCCTGCTGTCGTCTCC 3′
(SEQ ID NO: 15)
5′ACTGCCTGTCAATCCTGCTGTATCCTTATCGAATTAACTGCCTGTCC
CTCCTGCTGTCGTCCCC 3′
(SEQ ID NO: 16)
5′ACTGCCTGTCACCCCTGCTGTTTTCCCATCCCCATTACTGCCTGTCC
CACCTGCTGTCTCTTTT 3′
(SEQ ID NO: 17)
5′ACTGCCTGTCTATCCTGCTGTTAAAAACCTTTAAATACTGCCTGTCT
CCCCTGCTGTTACCATT 3′
(SEQ ID NO: 18)
5′ACTGCCTGTCTACCCTGCTGTCCTAAAAATTTATAAACTGCCTGTCC
AACCTGCTGTCCCCTTT 3′
(SEQ ID NO: 19)
5′ACTGCCTGTCAACCCTGCTGTAATCTGTGTTTATGTACTGCCTGTCC
CCCCTGCTGTCTCCCCT 3′
(SEQ ID NO: 20)
5′ACTGCCTGTCCATCCTGCTGTTTTCCTTTCTCATATACTGCCTGTCA
ATCCTGCTGTAATCCGT 3′
(SEQ ID NO: 21)
5′ACTGCCTGTCTATCCTGCTGTCGTCAAACTTATCATACTGCCTGTCC
CTCCTGCTGTCTACCCT 3′
(SEQ ID NO: 22)
5′ACTGCCTGTCAACCCTGCTGTGCTCATCCCTCATCAACTGCCTGTCC
CTCCTGCTGTCTCCAAT 3′
(SEQ ID NO: 23)
5′ACTGCCTGTCACCCCTGCTGTATTTTTCCAATCCGAACTGCCTGTCA
CCCCTGCTGTCTCTTAT 3′
(SEQ ID NO: 24)
5′ACTGCCTGTCCATCCTGCTGTGTCTTAACTCCCATAACTGCCTGTCC
ATCCTGCTGTAACCTTT 3′
(SEQ ID NO: 25)
5′ACTGCCTGTCAAACCTGCTGTCCACCCACTAAACCTACTGCCTGTCC
ATCCTGCTGTAATAATT 3′
(SEQ ID NO: 26)
5′ACTGCCTGTCAATCCTGCTGTACTCAAATAAATAATACTGCCTGTCA
ATCCTGCTGTTCCTCGT 3′
(SEQ ID NO: 27)
5′ACTGCCTGTCAATCCTGCTGTTTTACCCTCACAATTACTGCCTGTCA
ACCCTGCTGTCAATAAT 3′
(SEQ ID NO: 28)
5′ACTGCCTGTCAACCCTGCTGTACCTCTCACCTATCAACTGCCTGTCC
CCCCTGCTGTCTACCGT 3′.

All RNAs have modified 2′-F pyrimidines (2′F-Py) to improve stability and have been synthesized at the Synthetic and Biopolymer Chemistry Core, Beckman Research Institute, City of Hope, Duarte, CA. The sticky ends, consisting of 2′-F-Py and 2′-0-Methylpurine (2′OMe-Pu), are underlined. XXXX indicates the linear unsubstituted C4-C20 alkyl sequence acting as a linker. Alternatively, the sequence of the anti-axl aptamer may have a PEG at the 5′. To prepare the axl-miR-214sponge, axl-miR-214spongePEG and scramble-miR-214sponge complexes: 1) miR-214sponge sequence was incubated at 95°° C. for 10 minutes; 2) the aptamer with the sticky ends or scramble sequences were folded (5 min 85° C., 3 min on ice, 10 min at 37° C.); 3) equal amounts of aptamer/scramble and miR-214sponge were annealed by incubating them together at 37° C. for 30 minutes. Annealing efficiency was checked as described in (Catuogno et al. (2015). Selective delivery of therapeutic single strand antimiRs by aptamer-based conjugates. J Control Release 210: 147-159). In order to treat the cells with the axl, axl-miR-214sponge or scramble-miR-214sponge aptamers, the cells were plated in 24-well plates at 80% confluence and treated 24 hours later with the folded aptamers by adding them to their culture medium.

Protein or RNA Isolation, Western Blot, qRT-PCR

Total protein or RNA extracts, and the Western Blot (WB) and qRT-PCR assays were performed as described in Penna, E, Orso, F et al. (2011). “microRNA-214 contributes to melanoma tumour progression through suppression of TFAP2C”. EMBO J 30: 1990-2007.

### Proliferation, Migration, Invasiveness and Transendothelial Migration Assays

In vitro proliferation, migration, invasiveness and transendothelial migration assays were performed as described in (Penna, E, Orso, F et al. (2011). “microRNA-214 contributes to melanoma tumour progression through suppression of TFAP2C”. EMBO J 30: 1990-2007, Penna, E, et al. (2013) “miR-214 coordinates melanoma progression by upregulating ALCAM through TFAP2 and miR-148b downmodulation”, Cancer Res 73: 4098-4111, Cerchia et al. (2012). Targeting Axl with a high-affinity inhibitory aptamer. Mol Ther 20: 2291-2303).

### Mammosphere Formation Assays

Mammosphere formation assays were performed as described in the Technical Bulletin of Stem Cell Technologies Tumorsphere, DOCUMENT #29936 VERSION 1.1.0 JANUARY 2014 “Culture of Human Breast Cancer Cell Lines”, (https://www.stemcell.com/tumorsphere-culture-human-breast-cancer-cell-lines-lp.html) in 24-well plates coated with poly-HEMA (poly-2-hydroxyethylmethacrylate) using two different protocols. In one case, single breast tumor cells (8×10³ cells/well for the 4175-TGL cells, 1×10⁴ cells/well for the SKBR3 cells) were plated (day 0), maintained in suspension in MammoCult Medium (StemCell Technologies) and left untreated (controls =ctrl) or treated with 400 nmol/L of the anti-axl miR-214sponge or scr-miR-214sponge complex. Treatments were repeated on days 3 and 5 (200 nmol/L). On day 5, the size and number of the spheres were assessed by using a Zeiss AxioObserver microscope (Zeiss) and the ImageJ software (http://rsbweb.nih.gov/ij/).

For assessing the size, the long side (length) of the spheres was measured. For assessing the number, the total number of spheres was counted in 50 μl volume for each treatment.

### Histology and Immunohistochemistry

5 μm thick tissue sections were cut from formalin-fixed, paraffin-embedded (FFPE) tumor specimens and stained with Hematoxylin and Eosin (H&E) for standard histological observations. Immunohistochemical staining (IHC) was performed by using anti-KI67 or anti-cleaved caspase 3 antibodies, with avidin-biotin-peroxidase techniques (Anti-Mouse HRP-DAB Cell & Tissue Staining Kit, R & D Systems). The slides were counterstained with hematoxylin.

### Stability of the Axl-miR-214Sponge Chimeric Complex in Human Serum

The stability of the chimeric complex in human serum was assessed as described in (Catuogno et al. (2015). Selective delivery of therapeutic single strand antimiRs by aptamer-based conjugates. J Control Release 210: 147-159).

### In Vivo Tumor Growth and Metastasis Assays

All the experiments performed with animals were performed in compliance with ethics. NOD/SCID/IL2R_null (NSG) mice were injected with tumor cells as described in (Penna, E, et al. (2013) “miR-214 coordinates melanoma progression by upregulating ALCAM through TFAP2 and miR-148b downmodulation”, Cancer Res 73: 4098-4111, Orso, F, et al. (2016) “miR-214 and miR-148b Targeting Inhibits Dissemination of Melanoma and Breast Cancer”; Cancer Res 76: 5151-5162). Tumor cells were pre-treated 24 hours prior to injection with either the anti-axl aptamer or the axl-miR-214sponge aptamer. As soon as the tumors were palpable, they were treated with axl or miR-214sponge (400 pmol/injection, three injections per week). The mice were sacrificed and analyzed 23 days after the injections with the 4175-TGL cells. Alternatively, the mice were injected with 4175-TGL cells, and the animals were treated with an injection into the tail vein from Day 3 with PBS or axl-miR-214spongePEG (1600 pmol/injection, three injections per week) and sacrificed on Day 28. In both cases, the weight and morphology of the primary tumor and the lung or liver metastases were assessed as described in (Penna, E, et al. (2013) “miR-214 coordinates melanoma progression by upregulating ALCAM through TFAP2 and miR-148b downmodulation”, Cancer Res 73: 4098-4111, Orso, F, et al. (2016) “miR-214 and miR-148b Targeting Inhibits Dissemination of Melanoma and Breast Cancer”; Cancer Res 76: 5151-5162). Organ size (liver, spleen, kidney) (weight) and morphology (H & E) were analyzed at the end point.

### Statistical Analysis

All results are presented as the mean±Standard Deviation (SD) or ±Standard Error of the Mean (SEM), as indicated, and the two-tailed Student's t-test was used for comparisons. *=p<0.05; **=p<0.01; ***=p<0.001 were considered statistically significant. ns=indicates a non-statistically significant p-value.

### Results

The Axl-miR-214sponge Chimeric Complex and its Impact on miR-214 Expression

The therapeutic potential resulting from the modulation of miR-214 levels had previously been demonstrated (Orso, F, et al. (2016) “miR-214 and miR-148b Targeting Inhibits Dissemination of Melanoma and Breast Cancer”; Cancer Res 76: 5151-5162)., Dettori D, Orso F, Penna E, et al. (2018) “Therapeutic Silencing of miR-214 Inhibits Tumor Progression in Multiple Mouse Models”. Mol Ther.; 26 (8): 2008-2018). With the aim of delivering a sponge, i.e., a sequence designed to inhibit miR-214, specifically to breast tumor or melanoma cells, the inventors generated the chimeric axl-miR-214sponge complex (FFIG. 1a). In detail, a well-known RNA aptamer, GL21.T, was used, which is able to bind with great affinity and specificity to axl (Cerchia et al. (2012). Targeting Axl with a high-affinity inhibitory aptamer. Mol Ther 20: 2291-2303), i.e., an oncogenic receptor overexpressed on the surface of tumor cells and particularly on triple-negative breast tumor and melanoma cells (Quirico et al. (2020) “Axl-148b chimeric aptamers inhibit breast cancer and melanoma progression” IJBS 16: 1238-1251). To reduce steric hindrance and preserve proper folding of the complex, the original sponge DNA sequence consisting of 8 miR-214 binding sites, each 21 nucleotides in length, interspersed with 15 spacer nucleotides, perfectly complementary to the miR-214 seed region and with a mismatch in position 9-12

(Penna, E, Orso, F et al. (2011). “microRNA-214 contributes to melanoma tumour progression through suppression of TFAP2C”. EMBO J 30: 1990-2007, Orso, F, et al. (2016) “miR-214 and miR-148b Targeting Inhibits Dissemination of Melanoma and Breast Cancer”; Cancer Res 76: 5151-5162).), was shortened. In the new sequence there are two miR-214 binding sites separated by 15 nucleotides which act to give flexibility to the sequence and improve miR-214 binding (FIG. 1b). In addition, the terminal part of the miR-214 sponge contains 7 nucleotides and a sticky sequence that binds to the aptamer to obtain the chimeric complex.

The folding efficiency of the molecule was analyzed for each preparation by using a non-denaturing polyacrylamide gel: as shown, the chimera corresponds to the band at the top, followed by bands related to the miR-214 sponge and the anti-axl aptamer, indicating proper formation of the axl-miR-214sponge (FIG. 1c). In parallel, to assess in vitro serum stability, we incubated the chimeric complex with human serum at different time points starting from time zero (0 hours) up to 168 hours (FIG. 1d). After analyzing the non-denaturing polyacrylamide gel, the axl-miR-214sponge conjugate appeared to be stable for 8 hours, after which it began to degrade.

Subsequently, to assess the delivery of the miR-214 sponge to tumor cells expressing axl, breast tumor (4175-TGL 214) or melanoma (MA-2 214) cells overexpressing miR-214 were treated with the axl aptamer alone, the axl-miR-214sponge chimeric complex, an unrelated/scramble chimeric complex (scr-miR-214sponge), or were left untreated (ctrl). RNA was isolated 48 hours after treatments and miR-214 levels were measured by qRT-PCR. Alternatively, the cells were transfected with an anti-miR-214 (anti-214) or with the control sequence (anti-ctrl). A significant reduction in miR-214 levels was observed in the axl-expressing 4175-TGL 214 (FIG. 1e) or MA-2 214 (FIG. 1f) cells following treatment with the axl-miR-214sponge complex compared to the other treatments, and this modulation also occurred in the anti-214 transfected cells. Importantly, when axl-negative SKBR3 breast tumor cells were treated with the chimeric complex, no modulation of miR-214 was detected (FIG. 1g), but when the same cells were transfected with anti-214, miR-214 expression appeared to be reduced compared to the control. This indicates that the aptamer axl works selectively in delivering the miR-214 sponge into cells expressing axl. Even when miR-214 levels in SKBR3 cells were increased by transfection with a miR-214 precursor (pre-214) and the cells were treated as indicated above, no changes in miR-214 levels were observed (FIG. 1h).

### The Axl-miR-214sponge Complex Reduces the Motility of Breast Tumor and Melanoma Cells but has No Effect on In Vitro Proliferation

The inventors assessed whether axl-miR-214sponge is capable of acting on tumor spread, as miR-214 is a pro-metastatic miRNA (Penna, E, Orso, F et al. (2011). “microRNA-214 contributes to melanoma tumour progression through suppression of TFAP2C”. EMBO J 30: 1990-2007). Specifically, migration, invasiveness through Matrigel, and transendothelial migration through a HUVEC layer, simulating the extravasation process, were analyzed. To perform these experiments, the cells (4175-TGL 214, MA-2 214, and SKBR3) were either left untreated or were treated with the anti-axl aptamer alone (axl) or in combination with the miR-214 sponge (axl-miR-214sponge), with the sponge bound to a scramble aptamer (scr-miR-214sponge), or in parallel, the cells were transfected with anti-214 or its negative control (anti-ctrl).

Cell motility inhibition was observed in axl-positive 4175-TGL 214 breast tumor (FIG. 2a,c,e) and MA-2 214 melanoma (FIG. 2b, d, f) cells, but not in SKBR3 cells that do not express the axl receptor (FIG. 2g) compared to controls (ctrl or scr-miR-214sponge).

Aptamer axl alone also blocks the metastatic traits in axl-expressing cells, but the effect of the axl-miR-214sponge conjugate is enhanced due to the combination of the anti-axl aptamer and miR-214 sponge activities. Similar results were also observed in anti-214-transfected cells as compared to controls (anti-ctrl), in both cells expressing and not expressing the axl receptor. These data suggest that the biological effects obtained depend on the specific aptamer-mediated delivery and are specific to cells expressing axl on their surface. When proliferation was investigated in tumor cells, no significant change in treated or transfected cells was observed (FIG. 3a-f), confirming that miR-214 only affects cell movement. These data indicate the possibility of using the complex to block tumor cell motility and subsequent metastasization.

The Axl-miR-214Sponge Chimeric Complex Modulates the Levels of Both Direct and Indirect miR-214 Targets in Axl-Expressing Breast Tumor and Melanoma Cells

Since miRNA function is exerted by the negative regulation of their target genes, TFAP2γ and ITGα3, i.e., two direct miR-214 targets, were analyzed (Penna, E, Orso, F et al. (2011). “microRNA-214 contributes to melanoma tumour progression through suppression of TFAP2C”. EMBO J 30: 1990-2007). It was therefore investigated whether miR-214sponge could act on these two molecules when 4175-TGL 214, MA-2 214 or SKBR3 cells were treated with the anti-axl aptamer alone. with axl-miR-214sponge, with scr-miR-214sponge, or left untreated (ctrl) or, alternatively, transfected with anti-214 or anti-ctrl. As shown, axl-miR-214sponge increases TFAP2γ and ITGα3 protein expression compared to ctrl, anti-axl or scr-miR-214sponge in axl-expressing cells (FIG. 4a-b), whereas no effect occurred when SKBR3 cells were treated under the same conditions (FIG. 4c). In addition, all cell lines showed a reduction in target genes when transfected with anti-214 compared to anti-ctrl. In parallel, miR-214 coordinates a pathway that includes not only adhesion molecules (ITGα3) and transcription factors (TFAP2γ), but also miR-148b anti-metastatic miRNA and its direct targets such as ALCAM and ITGα5 (Penna, E, et al. (2013) “miR-214 coordinates melanoma progression by upregulating ALCAM through TFAP2 and miR-148b downmodulation”, Cancer Res 73: 4098-4111). axl-miR-214sponge is also able to act on indirect miR-214 targets, so when axl-positive cells were treated with the chimeric complex, a decrease in ALCAM protein levels was found (FIG. 4d-e), whereas no effect was detected in SKBR3 cells (FIG. 4f). These data indicate that axl-miR-214sponge works not only on direct miR-214 targets but also on the axis it forms with miR-148b.

### The Axl-miR-214sponge Chimeric Complex Reduces the Number and Size of the Mammospheres

A mammosphere assay was performed to test whether the axl-miR-214sponge conjugate can act on a 3D model. Specifically, single cells from the 4175-TGL 214 or the SKBR3 line were plated (Day 0) and treated on Days 0, 3 and 5 with aptamers axl, axl-miR-214sponge or scr-miR-214sponge, or were left untreated (ctrl), and the mammospheres were analyzed on Day 5 (FIG. 5a). When mammospheres derived from axl-expressing 4175-TGL 214 cells were examined on Day 5, significant reduction in the number (FIG. 5b) and size (FIG. 5c) thereof was observed in cells treated with axl-miR-214sponge compared to those treated with ctrl, axl or scr-miR-214sponge. In addition, when the above treatments were used on mammospheres derived from axl-negative SKBR3 cells, no change in number or size was detected (FIG. 5d-e). These data indicate that axl-miR-214sponge controls both formation and growth of the mammospheres and is able to penetrate even within 3D structures, suggesting the possibility that the chimeric complex may be able to enter the tumor mass to perform its anti-tumor function.

### The Axl-miR-214Sponge Chimeric Complex Blocks Tumor Cell Spread

To assess the efficacy of the axl-miR-214sponge chimeric complex on metastatic spread in mice, RFP-expressing 4175-TGL breast tumor cells pre-treated for 24 hours with either the anti-axl aptamer or axl-miR-214sponge were injected into the mammary gland of NOD/SCID/IL2R_null (NSG) immunocompromised mice, and axl or axl-miR-214sponge was injected into the tumor mass 3 times a week starting on day 2(FIG. 6a). Metastasis formation was analyzed 23 days after injection of the tumor cells, and a decrease in the number of lung metastases was observed (FIG. 6b) in mice treated with axl-miR-214sponge compared to those treated with axl. H&E tests on the primary mass showed an increase in necrosis (FIG. 6c), while immunohistochemistry tests on cleaved caspase-3 and KI67 showed an increase in apoptosis (FIG. 6d), but no changes in proliferation (FIG. 6e) in tumors treated with the chimeric complex compared to those injected with axl alone. Importantly, no toxicity was observed following the administration of the aptamer alone or conjugated with miR-214 sponge. In fact, no morphological or weight changes occur in the organs analyzed, i.e., the liver, spleen, and kidneys (FIG. 7a-c). In another experiment, 4175-TGL cells were injected, and the mice were injected into the tail vein from day 3 with PBS or axl-miR-214spongePEG (FIG. 8a), i.e., a complex that differs from axl-miR-214sponge in the addition of a PEG molecule at the 5′ that is intended to increase the weight of the chimeric complex and, consequently, reduce its renal filtration, thereby increasing its persistence in the circulation. Mice injected with the complex showed a reduction in lung metastases compared to mice injected with PBS, indicating that systemic administration of the compound is possible (FIG. 8b).

What is claimed is:

1. An isolated chimeric complex comprising:
a) an anti-axl aptamer comprising, from the 5′ end to the 3′ end;
(i) a single stranded nucleic acid sequence capable of binding to the tyrosine kinase receptor axl;
(ii) a linker element consisting of a linear unsubstituted alkyl chain containing 4 to 20 carbon atoms; and
(iii) a first sticky sequence consisting of a single stranded nucleic acid sequence 15 to 20 bases in length; and
b) an oligonucleotide containing a DNA sponge directed against miR-214, the oligonucleotide comprising from the 5′ end to the 3′ end:
(iv) a binding sequence consisting of a single stranded DNA sequence containing a plurality of miR-214 binding regions interspersed with one or more linking regions, and
(v) a second sticky sequence consisting of a single stranded DNA sequence of the same length in bases as the first sticky sequence identified in (iii) and complementary thereto.

2. The isolated chimeric complex according to claim 1, wherein the anti-axl aptamer is an RNA aptamer or a DNA aptamer.

3. The isolated chimeric complex according to claim 1, wherein the single stranded nucleic acid sequence capable of binding to the tyrosine kinase receptor axl identified in (i) is SEQ ID NO:1 or the DNA sequence corresponding thereto wherein each U (uridine) of SEQ ID NO:1 is replaced by a dU (deoxyuridine).

4. The isolated chimeric complex according to claim 1, wherein the first sticky sequence identified in (iii) is SEQ ID NO:2 or the DNA sequence corresponding thereto wherein each U (uridine) of SEQ ID NO:2 is replaced by a dU (deoxyuridine).

5. The isolated chimeric complex according to claim 1, wherein the second sticky sequence identified in (v) is SEQ ID NO:4.

6. The isolated chimeric complex according to claim 1, wherein the linear unsubstituted alkyl chain identified in (ii) contains 6 to 18 carbon atoms.

7. The isolated chimeric complex according to claim 1, wherein the binding sequence identified in (iv) is selected from the group consisting of SEQ ID NO:3, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO: 27, and SEQ ID NO:28.

8. The isolated chimeric complex according to claim 1, wherein one or more pyrimidine bases are replaced by the corresponding 2′-fluoropyrimidine.

9. The isolated chimeric complex according to claim , wherein one or more purine bases are replaced by the corresponding 2′-O-methylpurine.

10. A method for inhibiting or reducing migration, invasiveness and/or transendothelial migration of tumor cells expressing the tyrosine kinase receptor axl in a subject in need thereof, the method comprising administering to the subject the chimeric complex of claim 1.

11. The method according to claim 10, wherein the tumor cells are characterized by overexpression of the tyrosine kinase receptor axl and of miR-214.

12. The method according to claim 10, wherein the tumor cells are selected from the group consisting of melanoma, pancreatic tumor, stomach tumor, prostate tumor, lung tumor, breast tumor, and ovary tumor cells.

13. (canceled)

14. A pharmaceutical composition comprising the chimeric complex according to claim 1, and at least one pharmaceutically acceptable vehicle, excipient and/or diluent.

15. The pharmaceutical composition according to claim 14, wherein the pharmaceutical composition is in a form suitable for subcutaneous, intravenous, intra-arterial, intraperitoneal, intramuscular, intranasal, inhalatory or intra-tumor administration.

16-19. (canceled)


SEQUENCE LISTING (28 sequences)

SEQ ID NO: 1 - RNA, 34 nt, synthetic construct
  misc_feature 1..34: note = GL21.T
  source 1..34: mol_type = other RNA
atgatcaatcgcctcaattcgacaggaggctcac

SEQ ID NO: 2 - RNA, 17 nt, synthetic construct
  misc_feature 1..17: note = first adhesive sequence
  source 1..17: mol_type = other RNA
gtacattctagatagcc

SEQ ID NO: 3 - DNA, 64 bp, synthetic construct
  misc_feature 1..64: note = binding sequence with multiple anti-miR-214 sites
  source 1..64: mol_type = other DNA
actgcctgtccatcctgctgtcctcttcatcgtcaaactgcctgtcaaacctgctgtaataccc

SEQ ID NO: 4 - DNA, 17 bp, synthetic construct
  misc_feature 1..17: note = second adhesive sequence
  source 1..17: mol_type = other DNA
ggctatctagaatgtac

SEQ ID NO: 5 - RNA, 34 nt, synthetic construct
  misc_feature 1..34: note = GL21.T scramble
  source 1..34: mol_type = other RNA
ggcgctagaaccttctaagcgaatacattaccgc

SEQ ID NO: 6 - DNA, 81 bp, synthetic construct
  misc_feature 1..81: note = miR-214sponge
  source 1..81: mol_type = other DNA
actgcctgtccatcctgctgtcctcttcatcgtcaaactgcctgtcaaacctgctgtaatacccggctatctagaatgtac

SEQ ID NO: 7 - DNA, 64 bp, synthetic construct
  misc_feature 1..64: note = binding sequence
  source 1..64: mol_type = other DNA
actgcctgtctaacctgctgtaactaccaataccaaactgcctgtcaaccctgctgtcttttgt

SEQ ID NO: 8 - DNA, 64 bp, synthetic construct
  misc_feature 1..64: note = binding sequence
  source 1..64: mol_type = other DNA
actgcctgtccgtcctgctgtcccatctatttccttactgcctgtccaccctgctgtttaatcc

SEQ ID NO: 9 - DNA, 64 bp, synthetic construct
  misc_feature 1..64: note = binding sequence
  source 1..64: mol_type = other DNA
actgcctgtccctcctgctgtaacaaccttcaccgaactgcctgtccatcctgctgtatcatcc

SEQ ID NO: 10 - DNA, 64 bp, synthetic construct
  misc_feature 1..64: note = binding sequence
  source 1..64: mol_type = other DNA
actgcctgtcaatcctgctgtcactaatttcatccaactgcctgtcacccctgctgtctatccc

SEQ ID NO: 11 - DNA, 64 bp, synthetic construct
  misc_feature 1..64: note = binding sequence
  source 1..64: mol_type = other DNA
actgcctgtccgtcctgctgtatcttacttcatcaaactgcctgtccctcctgctgtccccccc

SEQ ID NO: 12 - DNA, 64 bp, synthetic construct
  misc_feature 1..64: note = binding sequence
  source 1..64: mol_type = other DNA
actgcctgtccgtcctgctgtcgtatcctaccttaaactgcctgtcccacctgctgtatccccc

SEQ ID NO: 13 - DNA, 64 bp, synthetic construct
  misc_feature 1..64: note = binding sequence
  source 1..64: mol_type = other DNA
actgcctgtccatcctgctgtcattactccccttatactgcctgtcaatcctgctgttattccc

SEQ ID NO: 14 - DNA, 64 bp, synthetic construct
  misc_feature 1..64: note = binding sequence
  source 1..64: mol_type = other DNA
actgcctgtccatcctgctgtctcccgtttctttttactgcctgtccctcctgctgtcgtctcc

SEQ ID NO: 15 - DNA, 64 bp, synthetic construct
  misc_feature 1..64: note = binding sequence
  source 1..64: mol_type = other DNA
actgcctgtcaatcctgctgtatccttatcgaattaactgcctgtccctcctgctgtcgtcccc

SEQ ID NO: 16 - DNA, 64 bp, synthetic construct
  misc_feature 1..64: note = binding sequence
  source 1..64: mol_type = other DNA
actgcctgtcacccctgctgttttcccatccccattactgcctgtcccacctgctgtctctttt

SEQ ID NO: 17 - DNA, 64 bp, synthetic construct
  misc_feature 1..64: note = binding sequence
  source 1..64: mol_type = other DNA
actgcctgtctatcctgctgttaaaaacctttaaatactgcctgtctcccctgctgttaccatt

SEQ ID NO: 18 - DNA, 64 bp, synthetic construct
  misc_feature 1..64: note = binding sequence
  source 1..64: mol_type = other DNA
actgcctgtctaccctgctgtcctaaaaatttataaactgcctgtccaacctgctgtccccttt

SEQ ID NO: 19 - DNA, 64 bp, synthetic construct
  misc_feature 1..64: note = binding sequence
  source 1..64: mol_type = other DNA
actgcctgtcaaccctgctgtaatctgtgtttatgtactgcctgtcccccctgctgtctcccct

SEQ ID NO: 20 - DNA, 64 bp, synthetic construct
  misc_feature 1..64: note = binding sequence
  source 1..64: mol_type = other DNA
actgcctgtccatcctgctgttttcctttctcatatactgcctgtcaatcctgctgtaatccgt

SEQ ID NO: 21 - DNA, 64 bp, synthetic construct
  misc_feature 1..64: note = binding sequence
  source 1..64: mol_type = other DNA
actgcctgtctatcctgctgtcgtcaaacttatcatactgcctgtccctcctgctgtctaccct

SEQ ID NO: 22 - DNA, 64 bp, synthetic construct
  misc_feature 1..64: note = binding sequence
  source 1..64: mol_type = other DNA
actgcctgtcaaccctgctgtgctcatccctcatcaactgcctgtccctcctgctgtctccaat

SEQ ID NO: 23 - DNA, 64 bp, synthetic construct
  misc_feature 1..64: note = binding sequence
  source 1..64: mol_type = other DNA
actgcctgtcacccctgctgtatttttccaatccgaactgcctgtcacccctgctgtctcttat

SEQ ID NO: 24 - DNA, 64 bp, synthetic construct
  misc_feature 1..64: note = binding sequence
  source 1..64: mol_type = other DNA
actgcctgtccatcctgctgtgtcttaactcccataactgcctgtccatcctgctgtaaccttt

SEQ ID NO: 25 - DNA, 64 bp, synthetic construct
  misc_feature 1..64: note = binding sequence
  source 1..64: mol_type = other DNA
actgcctgtcaaacctgctgtccacccactaaacctactgcctgtccatcctgctgtaataatt

SEQ ID NO: 26 - DNA, 64 bp, synthetic construct
  misc_feature 1..64: note = binding sequence
  source 1..64: mol_type = other DNA
actgcctgtcaatcctgctgtactcaaataaataatactgcctgtcaatcctgctgttcctcgt

SEQ ID NO: 27 - DNA, 64 bp, synthetic construct
  misc_feature 1..64: note = binding sequence
  source 1..64: mol_type = other DNA
actgcctgtcaatcctgctgttttaccctcacaattactgcctgtcaaccctgctgtcaataat

SEQ ID NO: 28 - DNA, 64 bp, synthetic construct
  misc_feature 1..64: note = binding sequence
  source 1..64: mol_type = other DNA
actgcctgtcaaccctgctgtacctctcacctatcaactgcctgtcccccctgctgtctaccgt